(19) United States
(12) Patent Application Publication
(10) Pub. No.: US 2025/0033019 A1
(43) Pub. Date: Jan. 30, 2025

(54) REACTOR FOR OXYGEN-FREE DIRECT CONVERSION OF METHANE AND METHOD FOR PREPARING ETHYLENE USING THE SAME

(71) Applicant: KOREA RESEARCH INSTITUTE OF CHEMICAL TECHNOLOGY, Daejeon (KR)

(72) Inventors: Yong Tae KIM, Daejeon (KR); Sung Woo LEE, Daejeon (KR); Jung Ho SHIN, Daejeon (KR); Seung Ju HAN, Daejeon (KR); Tsegay Gebrekidan GEBREYOHANNES, Daejeon (KR); Jin Ju LEE, Daejeon (KR)

(73) Assignee: KOREA RESEARCH INSTITUTE OF CHEMICAL TECHNOLOGY, Daejeon (KR)

(21) Appl. No.: 18/753,540

(22) Filed: Jun. 25, 2024

(30) Foreign Application Priority Data
Jul. 26, 2023 (KR) 10-2023-0097637

(51) Int. Cl. B01J 19/24; B01J 23/44; B01J 23/50; B01J 31/28; C07C 2/76

(52) U.S. Cl. CPC B01J 19/24; B01J 23/44; B01J 23/50; B01J 31/28; C07C 2/76; B01J 2219/0277; B01J 2219/0281; C07C 2523/44; C07C 2523/50; C07C 2531/06

(57) ABSTRACT

The present invention relates to a oxygen-free direct conversion of methane reactor and a method for producing ethylene using the same. More specifically, the invention provides a oxygen-free direct conversion of methane reactor and a method for producing ethylene from methane, wherein the reactor is selectively heated to save energy, prevent overheating with high responsiveness, and minimize coke formation, thereby achieving high methane conversion rate and high ethylene yield at a high reaction rate. The method also allows for the production of ethylene and aromatic compounds.

### CROSS REFERENCE TO RELATED APPLICATION

The present application claims priority based on Korean Patent Application No. 10-2023-0097637, filed on Jul. 26, 2023, the entire content of which is incorporated herein for all purposes by this reference.

### BACKGROUND OF THE DISCLOSURE

### 1. Field of the Disclosure

The present invention relates to a reactor for oxygen-free direct conversion of methane and a method for preparing ethylene using the same. More specifically, it relates to methane oxygen-free direct conversion reactors and methods for producing ethylene by directly converting methane, the main component of natural gas, in anaerobic or oxygen-free conditions.

### 2. Description of the Related Art

In recent years, efforts have been consistently made to convert methane (CH₄), obtainable from natural gas, shale gas, etc., into high value-added products such as transportation fuels or chemical raw materials. Representative examples of high value-added products obtainable from methane include olefins (ethylene, propylene, butylene, etc.) and aromatic compounds. Among the technologies for obtaining high value-added products from methane, the MTO (Methanol to Olefins) process, which produces light olefins through methanol from synthesis gas (H₂+CO) obtained by methane reforming, and the FTO (Fischer-Tropsch to Olefins) process, which directly produces light olefins from synthesis gas, are known as the most feasible technologies.

However, in such technologies for producing high value-added products via synthesis gas, additional hydrogen (H₂) or carbon monoxide (CO) is required as a reactant to remove oxygen atoms from carbon monoxide (CO), resulting in a decrease in the efficiency of hydrogen or carbon atom utilization in the overall process.

Therefore, there is a demand for new technologies that can directly convert methane into high value-added products without relying on synthesis gas. To directly convert methane into high value-added products, it is necessary to first activate methane by cleaving the strong C—H bonds (434 KJ/mol) present in methane. In this regard, research on methane activation using oxygen, known as the oxidative coupling of methane (OCM) reaction, has been actively pursued. However, in the OCM reaction, the vigorous reactivity of O₂ leads to the formation of thermodynamically stable H₂O and CO₂ in large quantities, which still poses a problem by reducing the efficiency of hydrogen or carbon atom utilization.

To address these issues, technologies for producing ethylene, aromatic compounds, etc., by the direct conversion of methane under anaerobic or oxygen-free conditions have been developed recently. However, due to the low reactivity of methane, these processes typically occur at high temperatures and pressures, necessitating the development of suitable reactors and catalysts. However, according to previous research results, the rapid deactivation of catalyst activity due to carbon n (coke) deposition under high temperature and high pressure conditions has emerged as a key issue (see non-patent documents 0001 and 0002).

In U.S. Pat. No. 4,424,401, a method is disclosed for aromatizing hydrocarbons to form hydrocarbon mixtures by diluting acetylene with inert gases, water, hydrogen, methane, and alcohols in the presence of a zeolite catalyst ZSM-5. In U.S. Pat. No. 8,013,196, a method is disclosed for producing ethylene by thermally converting methane-containing feedstock to produce acetylene-containing effluent, and then hydrogenating the resulting acetylene-containing effluent to produce ethylene.

However, in these prior art documents, methods for aromatizing methane or acetylene on zeolite or other catalysts also suffer from the problem of rapid deactivation of catalyst performance due to the accumulation of coke fragments and rapid polymerization of acetylene. Additionally, high levels of other by-products derived from acetylene conversion are formed.

In particular, the disclosed methods for producing ethylene from methane-containing feedstock have shown several disadvantages, such as catalyst deactivation, excessive hydrogenation, formation of green or oil carbon, temperature overheating, or low productivity per unit volume of reactor.

Therefore, there is a need in the art, particularly, for the development of improved methods and reactors that enable more efficient and stable production of ethylene from methane.

### RELATED PRIOR ART DOCUMENTS

### Patent Documents

(Patent Document 0001) U.S. Pat. No. 4,424,401 (Date of registration: 1984.01.03)
(Patent Document 0002) U.S. Pat. No. 8,013,196 (Date of registration: 2011.09.06)

### Non-Patent Documents

(Non-Patent Document 0001) X, Guo et al., Direct, Nonoxidative Conversion of Methane to Ethylene, Aromatics, and Hydrogen, Science, 344, 2014, 616˜619
(Non-Patent Document 0002) Mann Sakbodin et al., Hydrogen-Permeable Tubular Membrane Reactor: Promoting Conversion and Product Selectivity for Non-Oxidative Activation of Methane over an FeVSiO₂ Catalyst, Angew. Chem. 2016, 128, 16383˜16386

### SUMMARY OF THE DISCLOSURE

The main purpose of the present invention is to address the aforementioned problems. Specifically, in the production of ethylene from methane, the invention aims to provide a oxygen-free direct conversion of methane reactor and a method for producing ethylene and aromatic compounds using the same, which is selectively heats the reactor to save energy while preventing overheating with high responsiveness and minimizing coke generation, thereby providing a high reaction rate for high methane conversion and high ethylene yield.

To achieve the above objectives, in one embodiment of the present invention, a reactor for oxygen-free direct conversion of methane is provided, comprising an inlet for introducing a methane-containing feedstock, a reaction section for reacting the methane-containing feedstock introduced from the inlet to produce an ethylene-containing product, and an outlet for discharging the ethylene produced from the reaction section. The reaction section is made of FeCrAl alloy with an oxidation film formed on the inner surface, and a carbon layer is formed on the surface of the oxidation film of the FeCrAl alloy.

In a preferred embodiment of the present invention, the oxidation film can be an alumina film formed by oxidizing the inner surface of the reactor.

In a preferred embodiment of the present invention, the carbon layer can be a coke layer formed by the direct conversion reaction of methane within the reactor.

In a preferred embodiment of the present invention, the carbon layer can have a thickness ranging from 1 μm to 1 mm.

In a preferred embodiment of the present invention, the reaction section can be heated by one or more methods selected from resistance heating, discharge heating, induction heating, and dielectric heating.

In a preferred embodiment of the present invention, the reaction section can be divided into a first reaction zone for generating acetylene by the reaction of the methane-containing feedstock introduced from the inlet, and a second reaction zone for generating ethylene by hydrogenating the acetylene generated from the first reaction zone. The second reaction zone can be filled with an acetylene hydrogenation catalyst.

In a preferred embodiment of the present invention, the acetylene hydrogenation catalyst can comprise a catalyst support coated with sulfur-containing polymers on the surface, and a catalyst active component loaded on the catalyst support.

In a preferred embodiment of the present invention, the catalyst support can be selected from a group consisting of silica, alumina, magnesia, silica-alumina, silica-magnesia, zirconia, silicon carbide, and alumina-magnesia.

In a preferred embodiment of the present invention, the catalyst active component may be selected from a group consisting of gold, palladium, ruthenium, iron, nickel, cobalt, molybdenum, gold, silver, copper, titanium, gallium, cerium, aluminum, zinc, and lanthanum.

In a preferred embodiment of the present invention, the sulfur-containing polymers can be polymers comprising repeating units represented by chemical formulas 1 or 2.

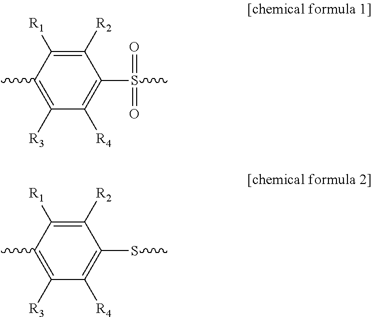

In chemical formulas 1 or 2, R₁ to R₄ can be the same or different from each other, and each independently represents hydrogen, alkyl groups of 1 to 10 carbon atoms, or aryl groups of 6 to 20 carbon atoms, wherein adjacent groups can combine to form hydrocarbon rings.

Another embodiment of the present invention provides a method for producing ethylene from methane using the methane oxygen-free direct conversion reactor described above.

According to the present invention, in the production of ethylene from methane, the reactor can be selectively and uniformly heated through electrical heating methods such as Joule heating or induction heating, thereby preventing overheating. This approach not only facilitates energy savings and system miniaturization, making it applicable to various reactors, but also minimizes coke formation and maximizes catalytic reaction rates, providing high methane conversion and ethylene yield even at low hydrogen supply rates.

### BRIEF DESCRIPTION OF DRAWINGS

FIG. 1 is a schematic cross-sectional view of a methane oxygen-free direct conversion reactor according to an embodiment of the present invention.

FIG. 2 is a schematic cross-sectional view of a methane oxygen-free direct conversion reactor according to another embodiment of the present invention.

FIG. 3 is a schematic diagram of a manufacturing process for an acetylene hydrogenation catalyst according to an embodiment of the present invention.

### DESCRIPTION OF THE PREFERRED EMBODIMENTS

Unless otherwise defined, all technical and scientific terms used herein have the same meaning as commonly understood by a skilled artisan in the field. Generally, the nomenclature used herein is well known and commonly used in the art.

Throughout the specification, whenever a portion is described as “comprising” or “including” certain components, it is intended to encompass additional components other than those specifically mentioned unless otherwise indicated.

The terms “comprising,” “including,” or “having,” among others, used in the specification refer to the presence of stated features, numerical values, steps, operations, components, parts, or combinations thereof, but do not preclude the presence or addition of one or more other features, numerical values, steps, operations, components, parts, or combinations thereof.

Throughout the specification, the terms “reaction zone” or “reaction area” refer to the space within the reactor where the reaction takes place, “inner” and “inside” refer to the direction towards the radial central axis of the reactor when the reactor is cut perpendicular to the direction of gravity, and “outer” or “outside” refer to the direction towards the circumference of the circle when the reactor is cut perpendicular to the direction of gravity.

Furthermore, throughout the specification, when components are distinguished by naming them as first, second, etc., it is for clarity of description and does not necessarily limit the components to any particular order as described below.

From one aspect, the present invention relates to a methane oxygen-free direct conversion reactor comprising an inlet for introducing a methane-containing feed, a reaction section for reacting the methane-containing feed introduced from the inlet to produce an ethylene-containing product, and an outlet for discharging the ethylene produced from the reaction section. The reaction section is divided into a first reaction zone that generates acetylene by reacting the methane-containing feed introduced from the inlet and a second reaction zone that hydrogenates the acetylene generated from the first reaction zone to produce ethylene. The reaction section is made of a FeCrAl alloy with an oxidized surface layer formed on the inner surface, with a carbon layer formed on the surface of the oxidized layer, characterizing a methane oxygen-free direct conversion reactor.

More specifically, in the production of ethylene from methane, the main factors inducing coke formation include catalyst cluster surfaces embedded in the reactor, stagnant flow of fluid, reactor material, heat transfer control, and reactor surface roughness.

Therefore, in the present invention, the reaction section of the reactor is composed of FeCrAl alloy heating elements with an oxidized surface layer formed thereon, and a carbon layer formed on the surface of the oxidized layer stabilizes the inner wall of the reactor by preventing overheating due to selectively uniform heating of the reactor with high responsivity, thereby enabling energy savings and system downsizing, and can be applied to various reactors. Furthermore, it minimizes coke formation and maximizes catalytic reaction rates, providing high methane conversion and ethylene yield even at low hydrogen supply rates.

The following detailed description of the present invention will now be provided with reference to the accompanying drawings.

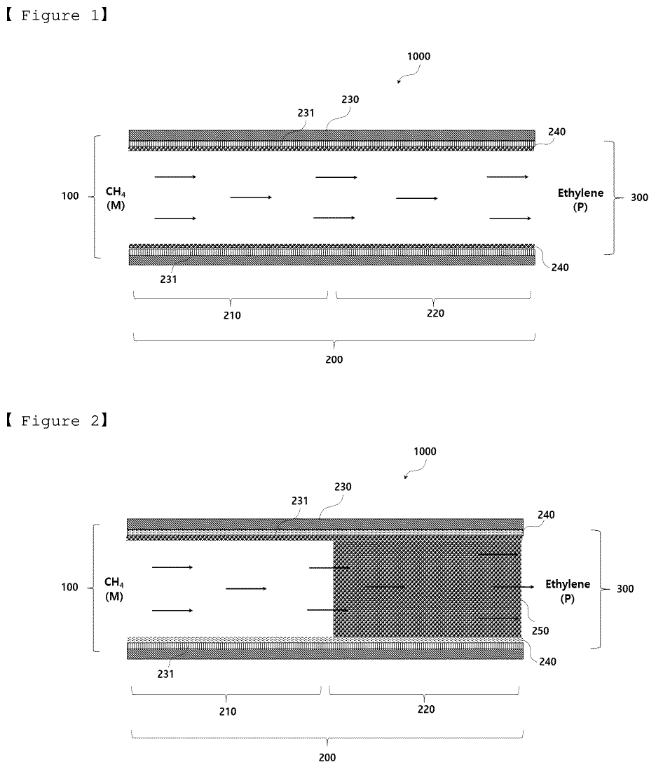

FIG. 1 is a schematic cross-sectional view of a methane oxygen-free direct conversion reactor according to an embodiment of the present invention, and FIG. 2 is a schematic cross-sectional view of a methane oxygen-free direct conversion reactor according to another embodiment of the present invention.

Referring to FIGS. 1 and 2, the methane oxygen-free direct conversion reactor (1000) according to the present invention includes an inlet (100) for introducing methane-containing feed (M), a reaction section (200) for generating ethylene-containing product (P) by reacting the methane-containing feed introduced from the inlet, and an outlet (300) for discharging the ethylene-containing product generated from the reaction section.

The configuration of the reactor (1000) may vary in dimensions or shape depending on production capacity, supply quantity, and catalyst, and can be adjusted by various methods known to those skilled in the art. Preferably, the reactor is tubular in shape, with an inlet (100) formed on one side for introducing methane-containing feed (M), and an outlet (300) formed on the opposite side for discharging the ethylene-containing product (P) after completion of the reaction.

The location of the inlet (100) of the reactor can be placed on the upper, lower, right, or left side of the reactor without limitation, and the outlet (300) can be positioned on the opposite side of the inlet corresponding to the inlet location.

The methane-containing feed (M) introduced through the inlet (100) can be any mixture containing methane, such as natural gas, and preferably, in addition to methane, it may contain inert gases and/or non-inert gases.

The methane content in the methane-containing feed may be 60% (v/v) or less of the total volume of the methane-containing feed introduced into the reactor, more preferably 18% (v/v) to 45% (v/v). The inert gases and/or non-inert gases may account for 40% (v/v) or more of the total volume of the methane-containing feed, more preferably 55% (v/v) or more.

The inert gases may include nitrogen, helium, neon, argon, krypton, and the non-inert gases may include carbon monoxide, hydrogen, carbon dioxide, water, mono-alcohols (carbon numbers 1-5), di-alcohols (carbon numbers 2-5), and alkanes (carbon numbers 2-8), and preferably may include inert gases such as nitrogen, hydrogen, oxygen, water, etc.

If hydrogen is included in addition to methane, the volume ratio of hydrogen to methane (H₂/CH₄) may be 1.1 to 6.0, preferably 1.1 to 5.1. If the volume ratio of hydrogen to methane (H₂/CH₄) is less than 1.1, it may lead to high partial pressure of aromatic compounds in the product and narrow operating range of the reactor (temperature, pressure, fluid flow, etc.) due to coke formation during the reaction. If the volume ratio of hydrogen to methane ((H₂/CH₄) exceeds 6.0, it may lead to low reactivity due to low methane partial pressure, resulting in increased energy cost due to increased reaction temperature.

The methane-containing feed introduced through the inlet (100) into the reaction section (200) generates the ethylene-containing product (P).

The reaction section (200) can be divided into a first reaction zone (210) where methane-containing feed introduced from the inlet reacts to generate acetylene, and a second reaction zone (220) where acetylene generated from the first reaction zone is hydrogenated to produce ethylene.

In the first reaction zone (210), the methane-containing feed introduced reacts to produce acetylene through thermal decomposition or methane oxygen-free direct conversion reaction as shown in Reaction 1.

2CH₄→C₂H₂+3H₂ [Reaction 1]

The reaction in the first reaction zone (210) can be performed at temperatures of 900° C. to 1,300° C., pressures below 10 bar, preferably at temperatures of 1,100° C. to 1,250° C. and pressures of 0.1 bar to 10 bar.

The reaction condition range is determined by considering the selectivity and yield of the desired hydrocarbons (such as acetylene), providing the advantage of maximizing the selectivity of methane to the desired hydrocarbons (such as acetylene). Under these conditions, coke formation is minimized, which helps to reduce pressure drop due to coke formation during the reaction and to minimize carbon efficiency loss caused by coke formation.

If the reaction temperature in the first reaction zone (210) is less than 900° C., the radical generation rate due to methane activation is low, resulting in low energy efficiency. If it exceeds 1,300° C., methane should minimize residence time in the reactor to suppress coke formation, leading to a high energy requirement for reactor heating.

Moreover, if the reaction pressure in the first reaction zone (210) exceeds 10 bar, coke formation is promoted, which can lead to issues requiring the reactor residence time and product cooling to be efficiently designed.

Furthermore, the gas hourly space velocity (GHSV) of the methane-containing feed in the first reaction zone can be from 500 h⁻¹ to 6,000 h⁻¹. If the space velocity is less than 500 h⁻¹, an issue may arise where the selectivity for coke in the product increases due to the promotion of C—C bond reactions of the primary products. Conversely, if the space velocity exceeds 6,000 h⁻¹, a problem may occur where the overall reactivity of the reactor decreases due to the lower reactivity of the catalyst surface, leading to a decline in reaction performance.

Additionally, the reaction section including the first reaction zone (210) is composed of FeCrAl alloy (230) with an oxidized surface layer (231) formed on the inner surface.

The FeCrAl alloy can be alloyed with iron 40 wt % to 80 wt %, chromium 19 wt % to 55 wt %, and aluminum 0.1 wt % to 10 wt %, and can further include carbon 0.08 wt % or less, silica 0.7 wt % or less, manganese 0.4 wt % or less, and other elements.

The aforementioned FeCrAl alloy is highly useful in various high-temperature applications due to its excellent oxidation resistance and is commonly used as an electrical resistance material. In particular, FeCrAl alloy has a melting point of 1,500° C., which is much higher than that of NiCr alloy. It exhibits resistance to electric current, making it suitable for resistance heating methods. Additionally, it can maintain high-temperature stability when applied to methane conversion, which consumes a significant amount of energy. This can also increase energy efficiency. Additionally, by oxidizing the surface of the FeCrAl alloy, an aluminum oxide layer can be formed on the alloy surface, providing excellent thermal stability even under oxidation and reduction conditions.

Therefore, in the present invention, by constructing the reaction section of the reactor with FeCrAl alloy (230), it is possible to perform the methane oxygen-free direct conversion reaction in a thermally stable manner while directly heating the reactor.

Various heating methods such as resistance heating, discharge heating, induction heating, and dielectric heating can be used to heat the reaction section. Through such heating methods, not only can the reaction section be selectively heated, but the heating intensity of the reaction section can also be easily adjusted by controlling the voltage and current applied to the reaction section. Furthermore, the reaction section made of FeCrAl alloy material can form an oxidized surface layer (231) on the contacting inner surface (inner wall) to increase the thermal stability of the reactor under reaction conditions and to uniformly form the carbon layer as described later.

The oxidized layer (231) can be formed by oxidizing the surface of the FeCrAl alloy, and the oxidation treatment can be applied without limitation if conditions for forming an oxide layer on the surface of the FeCrAl alloy are met. For example, oxidation can be performed in an air atmosphere at temperatures ranging from 500° C. to 1,050° C. for 8 to 20 hours, and the inflow rate of air can be applied without limitation as long as it allows oxidation of the reactor surface at high temperatures.

Moreover, the oxidized layer (231) can form a carbon layer (240) capable of catalytic surface reactions in the methane oxygen-free direct conversion, which can improve flow stagnation of reactants and products by coating the inner surface of the reactor rather than filling it, thereby increasing reaction rates by activating methane and inhibiting the formation of crystalline coke due to additional reactions.

The carbon layer (240) capable of inducing catalytic surface reactions in the oxygen-free direct conversion of methane can be formed on the oxidized layer (231). The carbon layer (240), which is not in a packed structure but rather coated on the inner surface of the reactor, is highly advantageous for forming laminar flow and can improve the flow of reactants and products, reducing stagnation. Consequently, it plays a role in the activation of methane on the catalyst surface, increasing the reaction rate, while simultaneously suppressing the formation of crystalline coke due to additional reaction processes.

The carbon layer (240) may be a coke layer formed by directly converting methane to non-oxidation in a reactor. The carbon layer (240) formed by the methane oxygen-free direct conversion reaction may vary in shape or structure along the reactor axis depending on the reactivity of methane, thereby affecting methane activity and additional reactivity. In particular, an increase in the surface roughness of the formed carbon layer can promote the conversion of radicals generated during the reaction into coke due to the high surface area provided. Therefore, the design of the carbon layer that can selectively increase the reaction rate of methane activation is necessary.

Furthermore, the carbon layer (240) can be formed with a thickness ranging from 1 μm to 1 mm. If the thickness of the carbon layer is less than 1 μm, there may be a problem where reactants react with the reactor surface instead of the carbon layer, increasing the selectivity of coke in the product. On the other hand, if the thickness exceeds 1 mm, the surface roughness of the carbon layer increases, which can act as scavengers for radicals generated under reaction conditions, also increasing the selectivity of coke in the product.

The carbon layer (240) can grow with low roughness and defects on oxide surfaces such as SiO₂ and Al₂O₃. Conversely, on metal surfaces, it can grow as crystalline carbon with high roughness and defects, promoting the conversion of reactants into coke.

The direct conversion reaction of methane to form a carbon layer in the reactor's FeCrAl alloy oxidation film can be applied without limitation if conditions for forming a coke layer on the oxidation film of the FeCrAl alloy are met. For example, it can occur at temperatures ranging from 900° C. to 2,000° C., pressures ranging from 0.1 bar to 10 bar, with methane-containing feed gas flow rates ranging from 255 cm/min to 1671 cm/min, and gas space velocities ranging from 76 h-1 to 501 h-1. The methane-containing feed gas may be the same as or different from the aforementioned methane-containing supply, and may also include inert gases and/or non-inert gases.

As previously described, the methane-containing feed gas undergoes a reaction in the first reaction zone (210) to produce acetylene, which then flows into the second reaction zone (220). In the second reaction zone (220), acetylene-containing reactants undergo hydrogenation to synthesize ethylene as shown in reaction 2. Hydrogen can be supplied along with methane as part of the methane-containing feed gas through the feed inlet (100) or can be additionally supplied to the second reaction zone (220) through hydrogen supply pipes (not shown).

C₂H₂+H₂→C₂H₄ [Reaction 2]

In the second reaction zone (220), hydrogenation can be performed at temperatures ranging from 30° C. to 900° C., pressures of less than 10 bar, preferably from 50° C. to 300° C., and pressures ranging from 0.1 bar to 5 bar. The weight hourly space velocity (WHSV) in the second reaction zone can range from 1.00×10³ mlg_{cat}⁻¹h⁻¹ to 1.00×10⁵ mlg_{cat}⁻¹h⁻¹, preferably from 1.00×10³ mlg_{cat}⁻¹h⁻¹ to 1.00×10⁴ mlg_{cat}⁻¹h⁻¹.

The reaction conditions are considered based on the selectivity and yield of hydrocarbons, with the advantage of maximizing the selectivity of acetylene to hydrocarbons. That is, under these conditions, coke formation is minimized, which in turn minimizes pressure drop and carbon deposition due to coke formation during the reaction.

If the reaction temperature in the second reaction zone (220) is below 30° C., the reactivity of acetylene is low, and the produced aromatic compounds may condense. Consequently, the reactor size of the second reaction zone (220) needs to be larger than that of the first reaction zone (210), leading to decreased catalyst reaction stability. On the other hand, if the temperature exceeds 900° C., side reactions of acetylene (dehydrogenation and coupling) dominate, which can result in an increased formation of coke.

Furthermore, if the reaction pressure in the second reaction zone (220) exceeds 10 bar, the side reactions of acetylene (dehydrogenation and coupling) and hydrogenation reactivity increase, consequently promoting coke formation. This can also result in a decline in catalyst performance due to the exothermic heat.

The weight hourly space velocity (WHSV) in the second reaction zone (220) can affect coke formation; WHSV below 1.00×10³ mlg_{cat}⁻¹h⁻¹ can promote coke formation, while WHSV exceeding 1.00×10⁵ mlg_{cat}⁻¹h⁻¹ can result in reduced reactivity of acetylene, requiring a larger reactor size compared to the first reaction zone (210).

Moreover, the second reaction zone (220) is also composed of FeCrAl alloy with an oxidized film (231) on the inner surface (inner wall), and a carbon layer is formed on the oxidized film. Inside the second reaction zone (220), an acetylene hydrogenation catalyst (250) can be packed to promote hydrogenation of acetylene.

The carbon layer formed in the second reaction zone is highly advantageous for the formation of laminar flow of reactants and products. It not only alleviates flow stagnation of reactants and products but also minimizes exothermic heat in the hydrogenation of acetylene, which is an exothermic reaction. This helps to suppress the formation of crystalline coke caused by strong reactant adsorption on the catalyst cluster surface.

The carbon layer in the second reaction zone, even when the acetylene hydrogenation catalyst is coated on the inner wall of the reactor to improve stability or performance, provides a high surface area on the inner wall of the reactor. This allows for a more stable increase in the loading rate of the acetylene hydrogenation catalyst compared to a reactor without a carbon layer.

The carbon layer in the second reaction zone can be formed on the inner surface of the reactor in the same manner as it was formed on the inner surface of the reactor in the first reaction zone.

In the second reaction zone, since a large amount of ethylene coexists with acetylene, ethylene consumption occurs during the acetylene removal process. Furthermore, rapid coke formation on the catalyst surface during the acetylene hydrogenation reaction can lead to rapid deactivation of the catalyst in a short time, and in cases where the catalyst has high activity for hydrogenation reactions, over-hydrogenation can lead to the predominant formation of ethane rather than ethylene in the product.

In the present invention, by filling the acetylene hydrogenation catalyst in the second reaction zone, the selectivity of ethylene can be improved, and coke formation can be prevented, allowing for stable operation of the reactor for a long period of time.

In one embodiment of the present t invention, the acetylene hydrogenation catalyst (250) may include a catalyst support (251) coated with sulfur-containing polymers (252) on the surface and containing catalytically active components (253) loaded into the catalyst support.

The catalyst support (251) may be any known in the art, such as silica, alumina, magnesia, silica-alumina, silica-magnesia, zirconia, silicon carbide, or alumina-magnesia, including one or more thereof, preferably alumina, but not limited thereto.

Furthermore, the catalyst support may be coated with sulfur-containing polymers (252) on the surface. The sulfur-containing polymer may be a polymer containing a polyphenylene sulfide structure, which may include repeating units represented by chemical formula 1 or chemical formula 2.

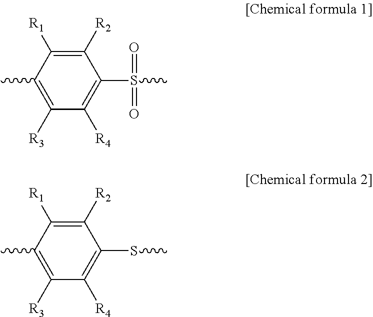

In the aforementioned chemical formulas 1 and 2, R₁ to R₄ may be the same as or different from each other, and each independently represents hydrogen, an alkyl group with 1 to 10 carbon atoms, or an aryl group with 6 to 20 carbon atoms, and adjacent groups may be bonded to each other to form a hydrocarbon ring.

Furthermore, in the aforementioned chemical formulas 1 and 2, ‘ [symbol] ’ denotes the point where repeating units are connected.

In one embodiment of the present invention, the alkyl groups in the aforementioned chemical formulas 1 and 2 can be straight-chain or branched-chain, and the number of carbon atoms is not specifically limited but is preferably from 1 to 10. Specific examples of alkyl groups include methyl, ethyl, propyl, isopropyl, butyl, n-butyl, isobutyl, tert-butyl, sec-butyl, 1-methylbutyl, 1-ethylbutyl, and the like, but are not limited thereto.

Moreover, in one embodiment of the present invention, specific examples of aryl groups in the aforementioned chemical formulas 1 and 2 include phenyl, biphenyl, terphenyl, quaterphenyl, naphthyl, anthracenyl, phenanthrenyl, pyrenyl, and the like, but are not limited thereto.

In a preferred embodiment of the present invention, R₁ to R₄ in the aforementioned chemical formulas 1 and 2 may all be hydrogen, and the hydrocarbon ring may be an aromatic hydrocarbon ring, with specific examples such as a benzene ring, but are not limited thereto.

Furthermore, in a more preferred embodiment of the present invention, the aforementioned chemical formula 1 may be represented by any one of the following chemical formulas 3 to 10.

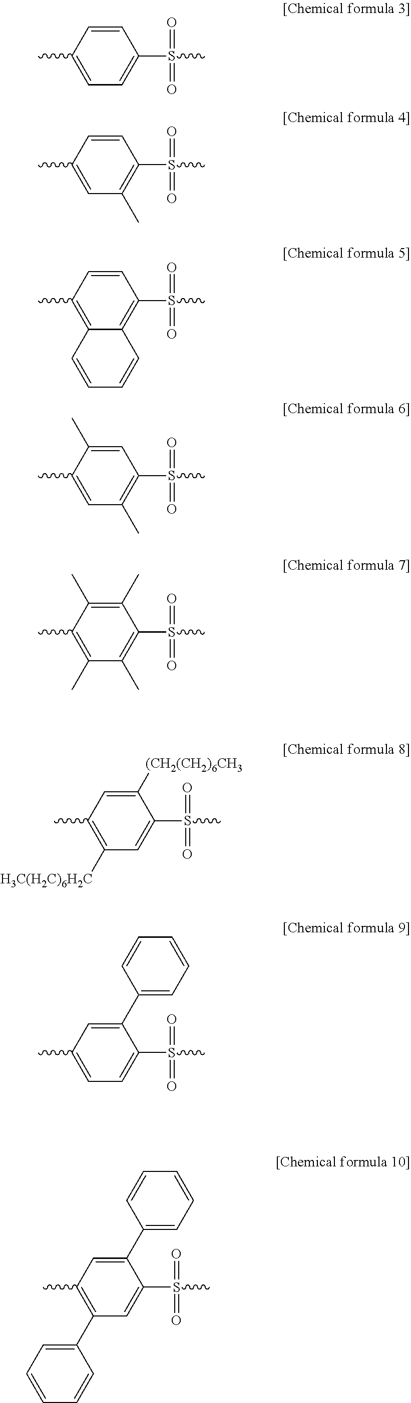

Furthermore, in a more preferred embodiment of the present invention, the aforementioned chemical formula 2 may be represented by any one of the following chemical formulas 11 to 18.

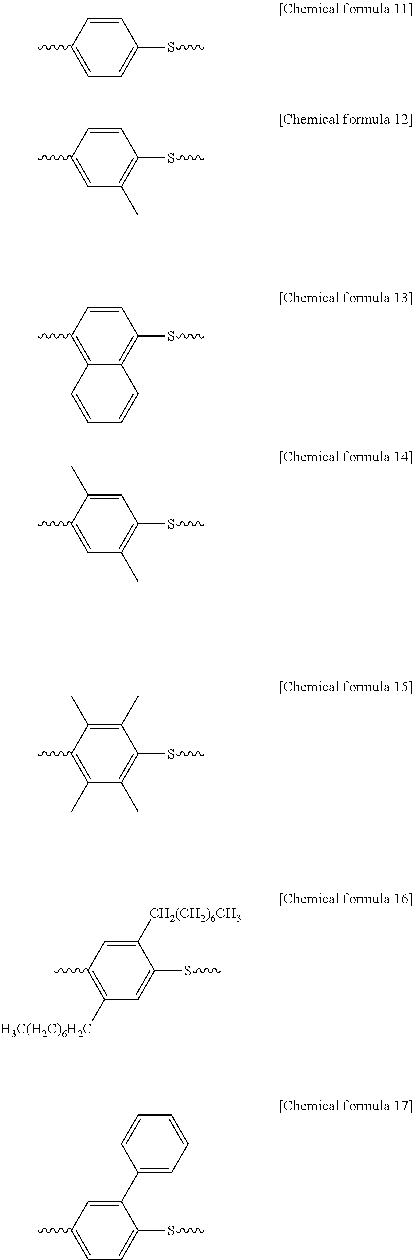

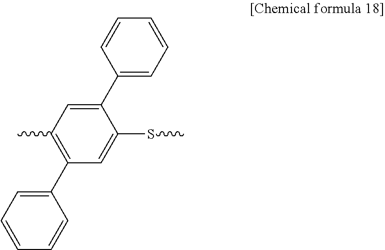

The average molecular weight of the sulfur-containing polymer may range from 1,000 g/mol to 100,000 g/mol, or specifically from 5,000 g/mol to 50,000 g/mol. If the average molecular weight of the sulfur-containing polymer is less than 1,000 g/mol, it is not desirable as it may lead to reduced catalytic stability, resulting in the inactivation of the catalyst due to polymer loss during the reaction. Conversely, if it exceeds 100,000 g/mol, the interaction with the catalyst support surface may be compromised, hindering smooth coating and potentially increasing the amount of polymer required for coating. The average molecular weight can be measured using Gel Permeation Chromatography (GPC) method.

The sulfur-containing polymer can be used commercially available polymers or can be synthesized by polycondensation of sulfur-containing organic monomers.

These sulfur-containing polymers typically have an amorphous-like structure, high thermal stability, and strong binding affinity with sulfur functional groups and catalytic active components, allowing for a high loading of catalytic active components while enabling selective ethylene reaction facilitated by sulfur components.

The content of the sulfur-containing polymer in the total weight of the acetylene hydrogenation catalyst can range from 0.1 wt % to 30 wt %. If the content is less than 0.1 wt %, the enhancement of reaction selectivity and catalyst stability is minimal, while exceeding 30 wt % may induce C—C coupling reactions due to over-hydrogenation, which is undesirable.

Moreover, the catalytic active component may include metals such as gold, palladium, ruthenium, iron, nickel, cobalt, molybdenum, silver, copper, titanium, gallium, cerium, aluminum, zinc, or lanthanum, with palladium and/or silver being preferable for ethylene hydrogenation activity.

The content of the catalytic active component in the total weight of the acetylene hydrogenation catalyst can range from 0.1 wt % to 50 wt %. If the content is less than 0.1 wt %, the catalytic activity sharply decreases, while exceeding 50 wt % may result in reduced dispersion of catalytic active components in the catalyst support, leading to decreased reactivity relative to the catalytic active component content.

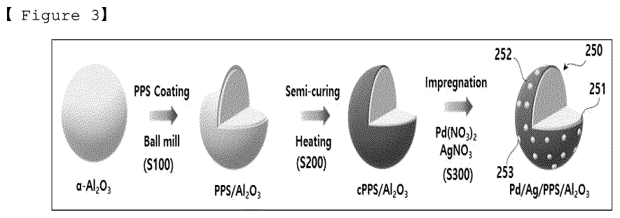

The method for preparing the acetylene hydrogenation catalyst (250) may include, in one embodiment as shown in FIG. 3, the steps of: (S100) at least partially coating the catalyst support with a sulfur-containing polymer; (S200) heat-treating the catalyst support coated with the sulfur-containing polymer; and (S300) loading the catalyst active component t onto the heat-treated catalyst support coated with the sulfur-containing polymer.

In the method for preparing the acetylene hydrogenation catalyst according to one embodiment of the present invention, the details regarding the catalyst support, sulfur-containing polymer, and catalyst active components are the same as described above.

In one embodiment of the present invention, the step of coating at least a portion of the catalyst support with the sulfur-containing polymer (S100) can be performed using known methods in the art, preferably by mechanically mixing the catalyst support with the sulfur-containing polymer.

The mechanical mixing can be ball milling, wherein the ball milling involves adjusting the weight ratio of the catalyst support and the sulfur-containing polymer to milling balls in the range of 1:80 to 1:120, loading them into a container, and performing the milling at a rotational speed of 120 rpm to 170 rpm for 0.1 to 2 hours. Through this mechanical mixing, the sulfur-containing polymer can be uniformly coated on the catalyst support with a thickness ranging from 1 μm to 100 μm.

As described above, the catalyst support coated with the sulfur-containing polymer can be heat-treated at 175° C. to 280° C. for 30 minutes to 5 hours to achieve semi-curing. If the heat treatment temperature is below 175° C., efficient cross-linking may not occur, while exceeding 280° C. may lead to the formation of low-active sulfite linkages due to excessive oxidation. The heat treatment time is preferably adjusted to 30 minutes to 5 hours to match the polymer's curing, but can be extended if necessary.

Subsequently, the heat-treated catalyst support coated with the sulfur-containing polymer is then loaded with the catalyst active component. The method for loading the catalyst active component is not particularly limited and can utilize methods known in the art. For example, a solution (loading solution) containing a compound as a precursor of the catalyst active component can be prepared, and the catalyst support can be impregnated with the prepared loading solution, or the reduced catalyst active component and the catalyst support can be stirred together for loading. The precursor of the catalyst active component can be a salt or complex of the catalyst active component. Specifically, it can be a water-soluble salt, such as acetate, nitrate, sulfate, carbonate, hydroxide, halide, or their hydrates, but it is not limited to these examples.

Meanwhile, the oxygen-free direct conversion of methane reactor according to the present invention may have a ratio of the mean residence time of the first reaction zone to that of the second reaction zone in the range of 1 to 30, preferably 3 to 30.

If the ratio of the mean residence time of the first reaction zone to that of the second reaction zone is less than 1, the size of the reactor for the second reaction zone relative to the first reaction zone may increase, leading to reduced thermal efficiency. Conversely, if it exceeds 30, the reactivity of acetylene is low, which may result in acetylene being present in the product.

The synthesized ethylene-containing product is discharged through the discharge part (130) of the reactor to the outside or downstream.

The present invention relates to a method for producing ethylene from methane using the oxygen-free direct conversion of methane reactor from a different perspective.

According to the method for producing ethylene according to the present invention, methane-containing feed (M) is supplied through the inlet (100) of the oxygen-free direct conversion of methane reactor (1000), acetylene is generated by the reaction in the first reaction zone (210) of the reactor, the generated acetylene flows into the second reaction zone (220), and the acetylene introduced into the second reaction zone (220) is hydrogenated by hydrogen to produce ethylene and aromatic compounds, which are then discharged through the discharge part (300).

Since the method for producing ethylene according to the present invention corresponds to the oxygen-free direct conversion of methane reactor mentioned above, those skilled in the art will be able to clearly understand the manufacturing method without further explanation to avoid redundancy.

Below, the present invention will be described more specifically through specific embodiments. The following embodiments are merely examples to aid in understanding the present invention, and the scope of the present invention is not limited thereto.

### Manufacturing Example 1

300 g of α-Al₂O₃ beads with an average diameter of 3 mm and 3 g of Poly(1,4-phenylene sulfide) powder with a number-average molecular weight of Mn˜10,000 and repeating units of Chemical Formula 11 were placed in a container and ball-milled at a speed of 200 rpm for 30 minutes (weight ratio of polymer to milling ball 1:100) to obtain PPS/Al₂O₃ coated with Poly(1,4-phenylene sulfide) with a thickness of 1 to 10 μm. The obtained PPS/Al₂O₃ was then placed in an alumina square crucible and heat-treated at 200° C. for 1 hour and 30 minutes to obtain cPPS/Al₂O₃ beads. The obtained cPPS/Al₂O₃ beads were subjected to wet impregnation with Pd(NO₃)₂·2H₂O (Sigma Aldrich, 99%) 0.76 g and AgNO₃ (Sigma Aldrich, 99%) 4.3 g. The mass of the precursor used was adjusted to achieve a mass ratio of Pd 0.1 wt % and Ag 0.9 wt %. Water used for wet impregnation was in a mass ratio of 1:1 with cPPS/Al₂O₃. After wet impregnation, the mixture was stirred at 70° C. for 1 hour at 50 rpm, and water was removed using a rotary evaporator to manufacture Pd/Ag/PPS/Al₂O₃ catalyst.

### Examples 1 to 4

### 1-1: Formation of Oxide Film

An alumina film was formed on a FeCrAl reactor (0.023 wt % C, 0.29 wt % Si, 0.13 wt % Mn, 21.1 wt % Cr, 5.7 wt % Al balanced in Fe, inner diameter: 2 cm) to stabilize the reactor. To achieve this, air was supplied into the FeCrAl reactor at a rate of 5 LPM to oxidize the reactor's interior. The linear velocity of the air was 1591 cm min⁻¹, and the air space velocity was 477.5 h⁻¹. The oxidation treatment was carried out by heating the FeCrAl reactor at a rate of 120° C. h⁻¹ to 1050° C. and treating the FeCrAl reactor in an air atmosphere for 8 hours to form an oxidation film on the inner surface of the reactor.

### 1-2: Formation of Carbon Layer

A carbon layer was formed on the FeCrAl reactor with the oxidation film formed in Example 1-1 to stabilize the reactor. For this purpose, methane-containing inlet gas was injected into the FeCrAl reactor with the oxidation film formed in Example 1-1 at 1000° C., adjusting the H₂/(CH₄+Ar) volume ratio to 1 to 3 to form a stable carbon layer at the reaction temperature up to 1150° C. Here, Ar was injected as an internal standard, and the linear velocity of the methane-containing inlet gas was adjusted to a range of 255 cm min⁻¹ to 1671 cm min⁻¹, and the space velocity was adjusted to a range of 76 h⁻¹ to 501 h⁻¹ to control the methane conversion rate to less than 5% and form a low defect carbon layer. Then the direct conversion reaction of methane was carried out by adjusting the flow rate to achieve an H₂/(CH₄+Ar) volume ratio of 3, a linear velocity of 3,819 cm·min⁻¹, and a space velocity of 1,145 h⁻¹, bringing the reactor to a temperature of 1,200° C. After completing the oxygen-free direct conversion of methane reaction, the carbon layer (thickness: 0.03 mm) formed on the inner wall of the reactor was examined using SEM (MIRA3 LMU, Tescan) to measure the carbon layer formed along the axial direction of the reactor. The length of the first reaction zone was 200 cm, and the length of the second reaction zone was 90 cm, and the reactor with an inner diameter of 2 cm was manufactured.

### Example 2

533 g of acetylene hydrogenation catalyst (Pd/Ag/PPS/Al₂O₃) manufactured in Example 1 was filled in the second reaction zone of the reactor manufactured in Example 1.

### Comparative Example 1

A reactor was manufactured by producing FeCrAl in the same manner as in Example 1, but without heat treatment at 1050° C. in an air atmosphere.

### Experimental Example 1: Evaluation of Oxygen-Free Direct Conversion of Methane Reaction

The oxygen-free direct conversion of methane reaction was measured using the reactors manufactured in Example 1 and Comparative Example 1. In this process, the methane-containing feed was a mixture of methane, hydrogen, and argon, with an H₂/(CH₄+Ar) volume ratio of 3, and the volume ratio of methane to argon fixed at 9:1. In Comparative Example 1, to reduce excessive coke selectivity at the H₂/(CH₄+Ar) volume ratio, the volume ratio of H₂/(CH₄+Ar) in the reaction gas was increased to 4, while all other conditions were kept the same for the experiment and product analysis. The reaction in the first reaction zone was conducted at 1,200° C. with a reaction pressure (Pₜₒₜₐₗ) of 1 bar. The methane-containing feed was injected at a space velocity of 1, 146 h⁻¹ and a linear velocity of 3,820 cm·min⁻¹ to perform the direct conversion of methane. During this process, the resistance heating of the reactor was used to heat a section of 200 cm, and the temperature of the second reaction zone was controlled between 12° and 180° C., with a weight hourly space velocity (WHSV) adjusted to 1, 350 ml·g_{cat}⁻¹ h⁻¹.

The gaseous hydrocarbon products obtained after the reaction were analyzed using an Agilent 8890 GC equipped with one Thermal Conductivity Detector (TCD) and two Flame Ionization Detectors (FID). Hydrogen, methane, argon, ethane, ethylene, and acetylene were separated on a Shincarbon ST 80/100 column and detected by the TCD. The conversion was calculated based on the area of methane relative to the area of argon, which was used as an internal standard. Hydrocarbons in the C1 to C6 range and benzene were separated on an Rt-Alumina BOND column and detected by the first FID. Aromatic compounds were separated on an RTx-VMS column and detected by the second FID. All gases were quantified using standard samples, and the results are shown in Table 1. Coke selectivity was calculated using the formula [S_{coke}=100−Σ product selectivity].

TABLE 1

| | | Comparative |
|---|---|---|
| | Example 1 | example 1 |
| Methane conversion (%) | 24.0 | 16.1 |
| Ethan selectivity (mol C %) | 3.1 | 1.3 |
| ethylene selectivity (mol C %) | 48.3 | 34.6 |
| Acetylene selectivity (mol C %) | 32.4 | 42.0 |
| C₃~C₄ selectivity (mol C %) | 3.3 | 3.0 |
| benzene selectivity (mol C %) | 10.6 | 3.1 |
| naphthalene selectivity (mol | 1.2 | 0.2 |
| C %) | | |
| Alkyl aromatics selectivity | 3.3 | 0.7 |
| (mol C %) | | |
| Coke selectivity (mol C %) | 1.0 | 15.8 |

As shown in Table 1, despite reducing the hydrogen content in the reactants, the reactor in Example 1 exhibited a decrease in coke selectivity and higher selectivity towards ethylene and benzene compared to the reactor in the Comparative Example 1.

<Experiment 2: Evaluation of Oxygen-Free Direct Conversion of Methane with Different Catalysts>

The oxygen-free direct conversion of methane reaction was measured using the same method as Experiment 1, but employing a reactor fabricated according to Example 2 under the conditions specified in Table 2.

TABLE 2

| | Experiment 2-1 | Experiment 2-2 |
|---|---|---|
| Volume fraction of methane- | 84:14.4:1.6 | 75.0:22.5:2.5 |
| containing feed (H₂:CH₄:Ar) | | |
| Linear velocity of first | 5,255 | 3,822 |
| reaction zone (cm · min⁻¹) | | |
| space velocity of first | 1,576 | 1,147 |
| reaction zone (h⁻¹) | | |
| Reaction temperature of | 1,270 | 1,200 |
| first reaction zone (° C.) | | |

TABLE 3

| | Experiment 2-1 | Experiment 2-2 |
|---|---|---|
| Methane conversion (%) | 56.57 | 21.85 |
| ethylene yield (%) | 40 | — |
| Ethylene production | — | 0.38 |
| rate (kg/day) | | |
| Ethan selectivity (mol C %) | 3.33 | 4.10 |
| ethylene selectivity (mol | 70.73 | 80.09 |
| C %) | | |
| Acetylene selectivity (mol | 6.63 | 1.56 |
| C %) | | |
| C₃~C₄ selectivity (mol C %) | 1.79 | 2.81 |
| benzene selectivity (mol C %) | 4.14 | 7.98 |
| Naphthalene selectivity (mol | 0.44 | 0.83 |
| C %) | | |
| Alkyl aromatics selectivity | 0.13 | 0.23 |
| (mol C %) | | |
| Coke selectivity (mol C %) | 12.81 | 2.24 |

As shown in Table 2, it was observed that the ethylene selectivity in both Experiment 2-1 and 2-2 was above 70%. Particularly, in Experiment 2-1, an ethylene yield of 40% was achieved, while in Experiment 2-2, it was confirmed that the production yield of ethylene could be secured at 0.38 kg/day. When using an electric furnace to carry out the reaction, it was confirmed that employing a catalyst in the second reaction zone, where the fluid in the first reaction zone is being cooled, can efficiently convert acetylene to ethylene.

While the invention has been described with reference to the disclosed embodiments and accompanying drawings, it should be understood that variations of different embodiments may be constructed within the scope and concept of the invention. Therefore, the scope of the invention is determined by the appended claims and their equivalents, and is not limited to the specific embodiments disclosed in this specification.

What is claimed is:

1. An oxygen-free direct conversion of methane reactor comprising:
an inlet for introducing methane-containing feedstock;
a reaction section for reacting the methane-containing feedstock introduced from the inlet to produce ethylene-containing product; and
an outlet for discharging the ethylene produced from the reaction section,
wherein the reaction section is made of FeCrAl alloy with an oxidation film formed on the inner surface, and a carbon layer is formed on the surface of the oxidation film of the FeCrAl alloy.

2. The oxygen-free direct conversion of methane reactor according to claim 1,
wherein the oxidation film is an alumina film formed by oxidizing the inner surface of the reactor.

3. The oxygen-free direct conversion of methane reactor according to claim 1,
wherein the carbon layer is a coke layer formed inside the reactor by the oxygen-free direct conversion of methane reaction.

4. The oxygen-free direct conversion of methane reactor according to claim 1,
wherein the carbon layer has a thickness ranging from 1 μm to 1 mm.

5. The oxygen-free direct conversion of methane reactor according to claim 1,
wherein the reaction section is heated by one or more methods selected from the group consisting of resistance heating, discharge heating, induction heating, dielectric heating, and electron beam heating.

6. The oxygen-free direct conversion of methane reactor according to claim 1,
wherein the reaction section is divided into a first reaction zone for generating acetylene by reacting the methane-containing feedstock and a second reaction zone for hydrogenating the generated acetylene to produce ethylene, and the second reaction zone is filled with an acetylene hydrogenation catalyst.

7. The oxygen-free direct conversion of methane reactor according to claim 6,
wherein the acetylene hydrogenation catalyst comprises a catalyst support coated with a sulfur-containing polymer on the surface and a catalyst active component loaded on the catalyst support.

8. The oxygen-free direct conversion of methane reactor according to claim 7,
wherein the catalyst support is selected from the group consisting of silica, alumina, magnesia, silica-alumina, silica-magnesia, zirconia, silicon carbide, and alumina-magnesia.

9. The oxygen-free direct conversion of methane reactor according to claim 7,
wherein the catalyst active component is selected from the group consisting of gold, palladium, ruthenium, iron, nickel, cobalt, molybdenum, gold, silver, copper, titanium, gallium, cerium, aluminum, zinc, and lanthanum.

10. The oxygen-free direct conversion of methane reactor according to claim 7,
wherein the sulfur-containing polymer is a polymer comprising repeating units represented by chemical formula 1 or chemical formula 2: 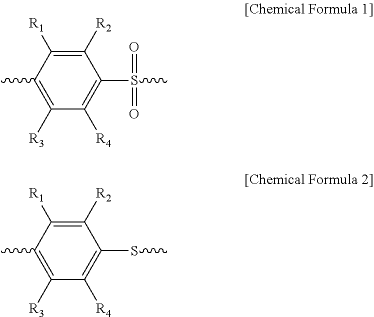
In Chemical Formula 1 or Chemical Formula 2, R₁ to R₄ are the same or different, and each independently represents hydrogen, alkyl group of 1 to 10 carbon atoms, or aryl group of 6 to 20 carbon atoms, or adjacent groups may be combined to form a hydrocarbon ring.

11. A method for producing ethylene from methane using the oxygen-free direct conversion of methane reactor according to claim 1.
